(19) United States
(12) Patent Application Publication
(10) Pub. No.: US 2010/0167997 A1
(43) Pub. Date: Jul. 1, 2010

(54) METHOD FOR STIMULATION COLLAGEN SYNTHESIS AND/OR KGF EXPRESSION

(75) Inventor: Sung-Hoon Kim, Seoul (KR)

Correspondence Address: SEED INTELLECTUAL PROPERTY LAW GROUP PLLC, 701 FIFTH AVE, SUITE 5400, SEATTLE, WA, 98104, US

(73) Assignee: ATYR PHARMA, INC., San Diego, CA (US)

(21) Appl. No.: 11/815,307

(22) PCT Filed: Jan. 24, 2006
(86) PCT No.: PCT/KR2006/000275

§ 371 Date: Oct. 26, 2009

(30) Foreign Application Priority Data
Feb. 1, 2005 (KR) PCTKR2005000300

(51) Int. Cl. A61K 38/16; A61P 17/02

(52) U.S. Cl. 514/12

(57) ABSTRACT

The present invention relates to a method for stimulating collagen synthesis and/or KGF expression, and more particularly, to a method for stimulating collagen synthesis and/or KGF expression using the AIMP1 or its fragment. The AIMP1 or its fragment can be effectively used for the stimulation of collagen synthesis and/or KGF expression in a subject in need thereof, the treatment of skin aging in the subject, the treatment of the flaccid and/or wrinkled skin in the subject, the promoting the smoothing and/or firming of the skin in the subject, the treatment of adverse cutaneous effects of menopause in the subject, and the treatment of adverse effects of menopause on the collagen.

This application claims priority to PCT Application No PCT/KR2005/000300, filed on Feb. 1, 2005, the contents of which are hereby incorporated by reference.

### TECHNICAL FIELD

The present invention relates to a method for stimulating collagen synthesis and/or KGF expression, and more particularly, to a method for stimulating collagen synthesis and/or KGF expression using the AIMP1 or its fragment.

### BACKGROUND ART

The entire human body is covered with the skin. Thus, the skin is considered to be the largest organ of the human body and consists generally of two layers, i.e., the epidermis and the dermis. The epidermis is the outermost and thinnest layer of the skin and has important functions to moisturize and protect the skin. More specifically, the epidermis protects the body from desiccation and the invasion of noxious substances, including UV light, virus and bacteria. Also, it naturally emulsifies oil in the sebaceous gland and water in the sweat gland so as to form a weakly acidic sebaceous membrane which protects the skin from the invasion of noxious substances and sterilizes bacteria. This epidermis consists mainly of keratinocytes and is maintained by a highly coordinated balance between the proliferation and differentiation of keratinocytes. The epidermal keratinocytes differentiate to form four layers consisting of the basal layer, the spinous layer, the granular layer and the horny layer. Particularly, the keratinocytes protect individuals from the external environment by a physical barrier, and produce and/or secrete substances regulating biological responses, including cytokines regulating immunological responses, thus regulating the immune response and inflammatory response of the skin. When the keratinocytes perform normal physiological functions, healthy and beautiful skin can be maintained.

The dermis functions to supply the epidermis with nutrients, to support to the epidermis, to protect the body from external damage, to store water and to regulate the body temperature. The dermis is composed of fibroblast cells and their extracellular matrix, and includes skin appendages therein, such as nerves, blood vessels, lymph, muscles, sebaceous glands, apocrine and eccrine. The fibroblast cells, which are the primary constituent in the structural assembly of the dermis, synthesize extracellular matrices, such as collagen, proteoglycan, other structural glycoproteins and etc. Among them, collagen is a fibrous protein forming 70% of the dermis, and functions to maintain the mechanical firmness (elasticity) of the skin, the cohesion of connective tissue and the adhesion of cells, and to induce cellular division and differentiation (Van der M. Rest et al., Biomaterials., 11:28-31, 1990; Shimokomaki M. et al., Ann. N.Y. Acad. Sci., 580:1-7, 1990; Van der Rest M. et al., Biochimie., 72(6-7):473-484, 1990). The collagen decreases with age and photoaging caused by exposure to UV irradiation, thus making the thinning of the skin and resulting in the formation of wrinkles in the skin (Artheu K. Balin et al., “Aging and the skin”, 1998). On the contrary, when the collagen metabolism is activated by the stimulation of collagen synthesis in the skin, the dermal matrix components will increase, leading to effects, such as the reduction of wrinkle formation, the improvement of skin firmness, and the strengthening of the skin.

The human skin is reduced in function by various internal and external factors with age. By active oxygen and the like produced by either the metabolic process of the skin cells or UV light, cellular components (e.g., lipids in the cellular membrane) are oxidized and their activity and biosynthesis are reduced. As a result, skin aging occurs. As the skin aging progresses, the epidermis thickness of the skin generally becomes thinner, and the dehydration of cells and tissues occurs, so that the cells and tissues are dried, fine wrinkles are increased and wrinkles become gradually deeper. Furthermore, the amount of collagen forming the bulk of the dermis and the activity of fibroblast cells synthesizing collagen are reduced, leading to a reduction in the synthesis of new collagen. As collagen is reduced in the dermis, the thickness of the dermis becomes smaller, resulting in the formation of wrinkles in the skin, the smoothness reductions and the firmness reductions of the skin. Accordingly, in order to effectively inhibit this skin aging, both the epidermal cells and the dermal cells should be activated and/or regenerated.

Meanwhile, AIMP1 (ARS-interacting multi-functional protein 1) was previously known as the p43 protein and renamed by the present inventors (Kim S. H. et al., Trends in Biochemical Sciences, 30:569-574, 2005). The AIMP1 is a protein consisting of 312 amino acids, which binds to a multi-tRNA synthetase complex to increase the catalytic activity of the multi-tRNA synthetase. The AIMP1 is highly expressed in microneuron in the resions of autoimmune diseases including encephalomyelitis, neuritis and uveitis in vitro. This phenomenon where the AIMP1 is highly expressed in a certain development stage and tissue suggests that the AIMP1 is related to inflammatory responses and cell apoptosis (Berger, A. C. et al., J. Immunother. 23:519-527, 2000). The present inventors have previously found that the AIMP1 and its N-terminal fragment can be used as effective cytokine, anti-cancer agents and angiogenesis inhibitors (Park H. et al., J. Leukoc. Biol., 71:223-230, 2002; Park S. G. et al., J. Biol. Chem., 277:45243-45248, 2002; Park H. et al., Cytokine, 21:148-53, 2002). However, the fact that the AIMP1 is involved in collagen synthesis and KGF expression is not yet found.

### DISCLOSURE OF THE INVENTION

Accordingly, it is an object of the present invention to provide a method for stimulating collagen synthesis and/or KGF (keratinocyte growth factor) expression in vitro or in vivo, a method for treating skin aging, a method for treating the flaccid and/or wrinkled skin, a method for promoting the smoothing and/or firming of the skin, a method for treating adverse cutaneous effects of menopause, and adverse effects of menopause on the collagen, each of the methods comprising administering to a subject in need thereof an effective amount of a polypeptide having the amino acid sequence set forth in SEQ ID NO: 1 or its fragment having the same physiological activity as the polypeptide.

Another object of the present invention is to provide a composition for stimulating collagen synthesis and/or KGF expression, and a cosmetic composition for preventing skin aging, each of the compositions comprising as an active ingredient a polypeptide having the amino acid sequence set forth in SEQ ID NO: 1 or its fragment having the same physiological activity as the polypeptide.

Still another object of the present invention is to provide uses of a polypeptide having the amino acid sequence set forth in SEQ ID NO: 1 or its fragment having the same physiological activity as the polypeptide, for preparing a composition for stimulating collagen synthesis and/or KGF expression, and a cosmetic composition for preventing skin aging, each of the composition comprising the polypeptide or its fragment as an active ingredient.

To achieve the above object, in one aspect, the present invention provides a method for stimulating collagen synthesis and/or KGF (keratinocyte growth factor) expression in vitro or in vivo, a method for treating skin aging, a method for treating the flaccid and/or wrinkled skin, a method for promoting the smoothing and/or firming of the skin, a method for treating adverse cutaneous effects of menopause, and adverse effects of menopause on the collagen, each of the methods comprising administering to a subject in need thereof an effective amount of one selected from the group consisting of:

(a) an isolated polypeptide having the amino acid sequence set forth in SEQ ID NO: 1;

(b) an isolated polypeptide having the amino acid sequence homology of at least 70% with the polypeptide (a);

and (c) a fragment of the polypeptide (a) or (b).

In another aspect, the present invention provides a composition for stimulating collagen synthesis and/or KGF expression, and a cosmetic composition for preventing skin aging, each of the compositions comprising as an active ingredient a polypeptide having the amino acid sequence set forth in SEQ ID NO: 1 or its fragment having the same physiological activity as the polypeptide.

In still another aspect, the present invention provides the uses of a polypeptide having the amino acid sequence set forth in SEQ ID NO: 1 or its fragment having the same physiological activity as the polypeptide, for preparing a composition for stimulating collagen synthesis and/or KGF expression, and a cosmetic composition for preventing skin aging, each of the composition comprising the polypeptide or its fragment as an active ingredient.

Hereinafter, the present invention will be described in detail.

The present invention is characterized by providing novel uses of the AIMP1, which are related to collagen synthesis and KGF (keratinocyte growth factor) expression.

The AIMP1 is a protein consisting of 312 amino acids. It is known that the AIMP1 is secreted from different types of cells, including prostate cancer cells, immune cells and transfect cells, and the secreted AIMP1 works on diverse target cells such as monocytes, macrophages, endothelial cells and fibroblast cells. The following three SNPs of the AIMP1 are known (see NCBI SNP database): substitution Of 79^{th} alanine (Ala) to proline (Pro) (SEQ ID NO: 24, SNP accession no. rs3133166); substitution of 104^{th} threonine (Thr) to alanine (Ala) (SEQ ID NO: 25, SNP accession no. rs17036670); and substitution of 117^{th} threonine (Thr) to alanine (Ala) (SEQ ID NO: 26, SNP accession no. rs2230255) in the amino acid sequence of the full-length AIMP1 (SEQ ID NO: 1).

Meanwhile, the present inventors hypothesized that, because the AIMP1 has various complex activities on various different target cells, the AIMP1 would use different structural motifs or domains for its diverse activities. To confirm this possibility, the present inventors determined the functional domain of AIMP1 for the stimulation of collagen synthesis and KGF (keratinocyte growth factor) expression by constructing the N-terminal fragment and C-terminal fragment of AIMP1 and then examining the activity of each of the fragments on the collagen synthesis and KGF (keratinocyte growth factor) expression (see Example 5). As a result, it was found that the C-terminal fragment of does not have activities of stimulation of collagen synthesis and KGF (keratinocyte growth factor) expression, but the N-terminal fragment has the activities (see FIG. 5).

Furthermore, in order to confirm which of the domains in the N-terminal fragment of AIMP1 has the above-described activity, the present inventors constructed deletion fragments and performed tests using the constructed fragments. As a result, it was found that the region of amino acids 6-46 of the AIMP1 has the activity to stimulate collagen synthesis and KGF expression (see FIGS. 6 and 7). From this test result, it could be found that fragments containing the region of amino acids 6-46 of AIMP1 and consisting of at least 192 amino acids, i.e., peptides selected from the group consisting of SEQ IN NO; 2, SEQ ID NO: 12, SEQ ID NO: 13 and SEQ ID NO: 14, have the activity to stimulate collagen synthesis and KGF expression. Also, it could be found that a fragment consisting of amino acids 1-192 of the AIMP1 (SEQ ID NO: 27) and a fragment consisting of amino acids 6-192 of the AIMP1 (SEQ ID NO: 18) have the activity to stimulate collagen synthesis and KGF expression (data not shown).

Collagen is a fibrous protein produced in dermal fibroblast cells and forming 70% of the dermis, and takes charge of the smoothing and firming of the skin. Thus, when the synthesis of collagen is reduced, skin aging will occur, and so the firming and smoothing of the skin will be rapidly reduced and as a result, the skin will be flaccid or wrinkled. On the contrary, when the metabolism of collagen is activated by the stimulation of collagen synthesis in the skin, the components of dermal matrices will be increased, leading to effects, such as wrinkle improvement, firmness improvement, and skin strengthening. The inventive AIMP1 or its fragment has the activity to stimulate the synthesis of collagen (see FIGS. 1 to 3 and 5 to 7).

KGF (keratinocyte growth factor) is a growth factor of keratinocytes that are the main components of the skin's epidermis. KGF is a paracrine factor produced from dermal fibroblast cells. KGF is involved in the regeneration and restitution of epithelial cells, and induces the growth of epithelial cells to regenerate the skin's epidermal layer (Moore T. et al, Lab. Invest. 60:237-244, 1989; Han D S et al., Am. J. Physiol. Gastrointest. Liver Physiol. 279:G1011-1022, 2000). Also, KGF is known also as fibroblast growth factor 7, a member of the fibroblast growth factor family, and transgenic rats introduced with a gene encoding KGF were shown to have thickened epidermis (Guo L. et al., EMBO J., 12(3):973-986, 1993). KGF is now registered as a cosmetic raw material in the Cosmetic, Toiletry and Fragrance Association. Also, it is known that KGF and collagen bind to each other to stimulate the division of keratinocytes (Ruehl M. et al., J Biol Chem., 277:26872-8, 2002). The AIMP1 and its fragment according to the present invention have the activity to stimulate the expression of KGF (see FIGS. 4, 5 and 6).

The AIMP1 and its fragment having the above-described activities can be used for the stimulation of collagen synthesis and/or KGF expression in the skin. Also, by stimulating collagen synthesis and/or KGF expression, the AIMP1 and its fragment can be used for the inhibition, treatment, improvement and/or prevention of skin aging or cutaneous signs caused thereby.

Accordingly, the present invention provides a method for stimulating collagen synthesis and/or KGF expression, a method for treating skin aging, a method for treating the flaccid and/or wrinkled skin, and a method for promoting the smoothing and/or firming of the skin, each of the methods comprising administering an effective amount of the AIMP1 or its fragment to a subject in need thereof.

Meanwhile, at menopause, the principal modifications regarding the dermis are reductions in the collagen level or in the dermal thickness. Studies related with postmenopausal changes in the collagen metabolism report the observation of reductions in bone density and skin thickness, and parallel decreases in the collagen level every year (C. Castelo-Braance et al., Maturitas., 18(3):199-206, 1994). Namely, after menopause, skin's collagen is most rapidly reduced in the first year of menopause, and reduced by 2.1% every year up to 20 years after menopause and by about 30% for 5 years after menopause. Thus, it is reported that various skin-aging phenomena, such as an increase in skin roughness and wrinkles, and a rapid reduction in skin firmness are accelerated, as the skin thickness rapidly decreases and sebum reduces. Also, it is suggested that the reduction of firmness caused by the reduction of collagen in the bladder wall is a major cause of increasing the frequency of urinary incontinence of postmenopausal women (Bergman A et al., Gynecol Obstet Invest., 37(1):48-51, 1994). Therefore, the inventive AIMP1 and its fragment having the activity to stimulate collagen synthesis and KGF expression can be used for the inhibition, treatment, improvement and/or prevention from skin aging in menopause, and abnormal conditions or symptoms caused in abnormal collagen metabolism in menopause, and the like. Accordingly, the present invention provides methods for treating adverse cutaneous effects of menopause and adverse effects of menopause on the collagen, each of the methods comprising administering an effective amount of the AIMP1 or its fragment to a subject in need thereof.

The AIMP1 used in the inventive methods may have an amino acid sequence set forth in SEQ ID NO: 1. The inventive AIMP1 includes functional equivalents thereof. As used herein, the term “functional equivalents” refers to polypeptides which exhibit substantially identical physiological activity to the AIMP1 having an amino acid sequence set forth in SEQ ID NO: 1.

The term “substantially identical physiological activity” refers to the activity to stimulate the synthesis of collagen and/or the expression of KGF. The functional equivalents may be polypeptides having a sequence homology of at least 70%, preferably at least 80%, and more preferably at least 90% to the amino acid sequence set forth in SEQ ID NO: 1. Preferably, the functional equivalents may be polypeptides of SEQ ID NO: 24, SEQ ID NO: 25 and SEQ ID NO: 26, which are known as SNPs of the AIMP1. More specifically, the term “functional equivalents” refers to peptides comprising the amino acid sequence having at least 70% amino acid sequence homology (i.e., identity), preferably at least 90%, and more preferably at least 95% for example, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% to the amino acid sequence of SEQ ID NO: 1 as a result of the addition, substitution or deletion of some amino acid of SEQ ID NO: 1 and that exhibit substantially identical physiological activity to the peptide of SEQ ID NO: 1. Sequence identity or homology is defined herein as the percentage of amino acid residues in the candidate sequence that are identical with amino acid sequence of SEQ ID NO: 1, after aligning the sequences and introducing gaps, if necessary, to achieve the maximum percent sequence identity, and not considering any conservative substitutions (as described above) as part of the sequence identity. None of N-terminal, C-terminal, or internal extensions, deletions, or insertions into the amino acid sequence of SEQ ID NO: 1 shall be construed as affecting sequence identity or homology. Thus, sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a predetermined portion of one or both sequences). The programs provide a default opening penalty and a default gap penalty, and a scoring matrix such as PAM 250 (a standard scoring matrix; see Dayhoff et al., in Atlas of Protein Sequence and Structure, vol. 5, supp. 3 (1978)) can be used in conjunction with the computer program. For example, the percent identity can be calculated as: the total number of identical matches multiplied by 100 and then divided by the sum of the length of the longer sequence within the matched span and the number of gaps introduced into the longer sequences in order to align the two sequences. The scope of the functional equivalents as used herein also encompasses derivatives obtained by modifying a part of the chemical structure of the inventive polypeptide while maintaining the basic framework and physiological activity of the polypeptide. For example, this includes structural modifications for altering the stability, storage, volatility or solubility of the peptide.

Moreover, a fragment of the AIMP1 may also be used in the present invention. The fragment of the AIMP1 may be a partial fragment of a polypeptide having the amino acid sequence set forth in SEQ ID NO: 1 or a polypeptide having a sequence homology of at least 70% to the amino acid sequence of SEQ ID NO: 1. Also, the fragment has an identity of 100% with a part of the amino acid sequence of the AIMP1 and shows substantially identical physiological activity as the inventive AIMP1. The fragment may consist of at least 10, preferably at least 41, and more preferably 100 contiguous amino acids selected from the amino acid sequence of SEQ ID NO: 1 or the amino acid sequence having a homology of at least 70% with the amino acid sequence of SEQ ID NO: 1. The inventive fragment may preferably be a peptide that comprises the amino acid sequence (SEQ ID NO: 12) consisting of a sequence of at least 41 contiguous amino acids selected among the amino acid sequence of AIMP1 (SEQ ID NO: 1) and that consists of at least 192 amino acids. More preferably, the inventive fragment may be a peptide consisting of any one amino acid sequence selected from the group consisting of SEQ ID NO: 2, SEQ ID NO: 12, SEQ ID NO: 13, SEQ ID NO: 14 and SEQ ID NOS: 27 to 34. The amino acid sequence of SEQ ID NOS: 29 to 31 are SNPs of the peptide having the amino acid sequence of SEQ ID NO: 27, and the amino acid sequence of SEQ ID NOs: 32 to 34 are SNPs of the peptide having the amino acid sequence of SEQ ID NO: 28.

The inventive polypeptide may be constructed by a genetic engineering method. For this purpose, a DNA molecule encoding the AIMP1 or its fragment is first constructed according to any conventional method. The DNA molecule may synthesized by performing PCR using suitable primers. Alternatively, the DNA molecule may also be synthesized by a standard method known in the art, for example using an automatic DNA synthesizer (commercially available from Biosearch or Applied Biosystems). The constructed DNA molecule is inserted into a vector comprising at least one expression control sequence that is operatively linked to the DNA sequence so as to control the expression of the DNA molecule, and host cells are transformed with the resulting recombinant expression vector. The transformed cells are cultured in a medium and condition suitable to express the DNA sequence, and a substantially pure polypeptide encoded by the DNA sequence is collected from the culture medium. The collection of the pure polypeptide may be performed using a method known in the art, for example, chromatography. In this regard, the term “substantially pure polypeptide” means the inventive polypeptide that does not substantially contain any other proteins derived from host cells. For the genetic engineering method for synthesizing the inventive polypeptide, the reader may refer to the following literatures: Maniatis et al., Molecula Cloning: A Laboratory Manual, Cold Spring Harbor Laboratory 1982; Sambrook et al., supra; Gene Expression Technology, Method in Enzymology, Genetics and Molecular Biology, Method in Enzymology, Guthrie & Fink (eds.), Academic Press, San Diego, Calif. 1991; and Hitzeman et al., J. Biol. Chem., 255, 12073-12080 1990.

Alternatively, the inventive peptide can be chemically synthesized according to any technique known in the art (Creighton, Proteins: Structures and Molecular Principles, W.H. Freeman and Co., NY 1983). Namely, the inventive peptide can be prepared by conventional step-wise liquid or solid phase synthesis, fragment condensation, F-MOC or T-BOC chemistry (Chemical Approaches to the Synthesis of Peptides and Proteins, Williams et al., Eds., CRC Press, Boca Raton Fla., 1997; A Practical Approach, Atherton & Sheppard, Eds., IRL Press, Oxford, England, 1989).

It is particularly preferred to use the solid phase synthesis to prepare the inventive peptide. The inventive peptide can be synthesized by performing the condensation reaction between protected amino acids by the conventional solid-phase method, beginning with the C-terminal and progressing sequentially with the first amino acid, the second amino acid, the third amino acid, and the like according to the identified sequence. After the condensation reaction, the protecting groups and the carrier connected with the C-terminal amino acid may be removed by a known method such as acid decomposition or aminolysis. The above-described peptide synthesis method is described in detail in the literature (Gross and Meienhofer's, The peptides, vol. 2, Academic Press, 1980).

Examples of a solid-phase carrier, which can be used in the synthesis of the peptide according to the present invention, include polystyrene resins of substituted benzyl type, polystyrene resins of hydroxymethylphenylacetic amide form, substituted benzhydrylpolystyrene resins and polyacrylamide resins, having a functional group capable of bonding to peptides. Also, the condensation of amino acids can be performed using conventional methods, for example dicyclohexylcarbodiimide (DDC) method, acid anhydride method and activated ester method.

Protecting groups used in the synthesis of the inventive peptide are those commonly used in peptide syntheses, including those readily removable by conventional methods such as acid decomposition, reduction or aminolysis. Specific examples of such amino protecting groups include formyl; trifluoroacetyl; benzyloxycarbonyl; substituted benzyloxycarbonyl such as (ortho- or para-) chlorobenzyloxycarbonyl and (ortho- or para-) bromobenzyloxycarbonyl; and aliphatic oxycarbonyl such as t-butoxycarbonyl and t-amyloxycarbonyl. The carboxyl groups of amino acids can be protected through conversion into ester groups. The ester groups include benzyl esters, substituted benzyl esters such as methoxybenzyl ester; alkyl esters such as cyclohexyl ester, cycloheptyl ester or t-butyl ester. The guanidino moiety may be protected by nitro; or arylsulfonyl such as tosyl, methoxybenzenesulfonyl or mesitylenesulfonyl, even though it does not need a protecting group. The protecting groups of imidazole include tosy, benzyl and dinitrophenyl. The indole group of tryptophan may be protected by formyl or may not be protected.

Deprotection and separation of protecting groups from carriers can be carried out using anhydrous hydrofluoride in the presence of various scavengers. Examples of the scavengers include those commonly used in peptide syntheses, such as anisole, (ortho-, meta- or para-) cresol, dimethylsulfide, thiocresol, ethanediol and mercaptopyridine.

The recombinant peptide prepared by the genetic engineering method or the chemically synthesized peptide can be isolated and purified according to methods known in the art, including extraction, recrystallization, various chromatographic techniques (e.g., gel filtration, ion exchange, precipitation, adsorption, reverse phase, etc.), electrophoresis and counter current distribution.

As used herein, the term “effective amount” refers to an amount showing an effect selected from the group consisting of the stimulation of collagen synthesis and/or KGF expression in vitro or in vivo, the treatment of skin aging, the treatment of the flaccid or wrinkled skin, the promoting the smoothing or firming of the skin; the treatment of adverse cutaneous effects of menopause, and adverse effects of menopause on collagen.

As used herein, the term “subject” means mammals, particularly, mammals including human beings, or the skin cells or skin tissues of mammals. The subject may be a patient in need of treatment. Also, the skin cells are preferably fibroblast cells.

The inventive AIMP1 and its fragment may be administered until the desired effect among the above-described effects is achieved. The inventive AIMP1 and its fragment may be administered by various routes according to any method known in the art. Namely, it may be administered by oral or parenteral routes, for example, oral, intramuscular, intravenous, intracutaneous, intraarterial, intramarrow, intrathecal, intraperitoneal, intranasal, intravaginal, intrarectal, sublingual and subcutaneous routes, or administered to gastrointestinal tracts, mucosae or respiratory organs. For example, the inventive polypeptide may be administered by a method of applying the polypeptide directly to the skin or a method comprising formulating the polypeptide in an injectable form, and then, injecting a given amount of the formulation into a subcutaneous layer with a 30-gauge injection needle or lightly pricking the skin with an injection needle. Preferably, the inventive polypeptide may be applied directly to the skin. Also, the inventive AIMP1 or its fragment may also be administered in a form bound to a molecule causing a high-affinity binding to a target cell or tissue (e.g., skin cell or skin tissue) or in a form encapsulated in the molecule. The inventive AIMP1 or its fragment can be bound to sterol (e.g., cholesterol), a lipid (e.g., a cationic lipid, virosome or liposome), or a target cell-specific binding agent (e.g., a ligand recognized by target cell specific receptor) using the technology known in the art. Suitable coupling agents or crosslinking agents may include, for example, protein A, carbodiimide, and N-succinimidyl-3-(2-pyridyidithio)propionate (SPDP).

The inventive AIMP1 or its fragment may be used as an active ingredient of a pharmaceutical, dermatological or cosmetic composition for the above-described uses.

The composition may be in any form known in the art. Preferably, it may be formulated in a form for application to the skin. Examples of this formulation include, but are not limited to, solution, suspension, emulsion, paste, gel, cream, lotion, powder, soap, surfactant-containing cleansing, oil, powder foundation, emulsion foundation, wax foundation and spray formulations. More preferably, the composition may be formulated in the form of softening lotion, nutrient lotion, nutrient cream, massage cream, essence, eye cream, cleansing cream, cleansing foam, cleansing water, packs, spray or powder.

When a formulation of the inventive composition is paste, cream or gel, usable carrier components may include animal oil, vegetable oil, wax, paraffin, starch, tragacanth, cellulose derivatives, polyethylene glycol, silicone, bentonite, silica, talc and zinc oxide. Also, when a formulation of the inventive composition is powder or spray, usable carrier components may include lactose, talc, silica, aluminum hydroxide, calcium silicate and polyamide powder, and particularly in the case of spray, it may additionally contain a propellant, such as chlorofluorohydrocarbon, propane/butane or dimethyl ether. When a formulation of the inventive composition is suspensions, usable carrier components may include liquid diluents, such as water, ethanol or propylene glycol, suspending agents, such as ethoxylated isostearyl alcohol, polyoxyethylene sorbitol ester and polyoxyethylene sorbitan ester, microcrystalline cellulose, aluminum metahydroxide, bentonite, agar, and tragacanth. Also, when a formulation of the inventive composition is surfactant-containing cleansing preparation, usable carrier components include aliphatic alcohol sulfate, aliphatic alcohol ether sulfate, sulfosuccinic monoester, isethionate, imidazolinium derivatives, fatty acid amide ether sulfate, alkylamidobetaine, aliphatic alcohol, fatty acid glyceride, fatty acid diethanolamide, vegetable oil, lanolin derivatives and ethoxylated glycerol fatty acid ester.

Also, the composition according to the present invention may be prepared into an injectable formulation for mesotherapy. The injectable formulation may be prepared using suitable dispersing or wetting agents and suspending agents according to the technology known in the art. For example, the inventive polypeptide may be formulated for injection by dissolution in saline or buffer.

Moreover, the inventive composition may also be prepared into formulations for oral administration. For oral administration, the polypeptide according to the present invention may be mixed with excipients to prepare formulations, such as ingestible tablets, buccal tablets, troches, capsules, elixirs, suspensions, syrups and wafers. These formulations may contain, in addition to the active ingredient, diluents (e.g., lactose, dextrose, sucrose, mannitol, sorbitol, cellulose and/or glycin) and lubricants (e.g., silica, talc, stearic acid and its magnesium salt or calcium salt and/or polyethylene glycol). The tablets may contain binders, such as magnesium aluminum silicate, starch paste, gelatin, tragacanth, methyl cellulose, sodium carboxymethylcellulose and/or polyvinyl pyrrolidone, and if necessary, may additionally contain disintegrants, such as starch, agar, alginic acid or its sodium salt, absorbing agents, coloring agents, flavoring agents and/or sweetening agents. These formulations may be prepared by a conventional mixing, granulation or coating method.

Also, in order to further enhance the effects of the present invention, the inventive composition may additionally contain known substances having the effects of the stimulation of collagen synthesis, the stimulation of fibroblast proliferation, the inhibition/improvement of skin aging, the moisturization of the skin, the increase of skin firmness/softness, the improvement of wrinkles, and the enhancement of skin functions. These substances may include, but are not limited to, retinoic acid, TGF (trans-forming growth factor), betulinic acid, cinnamic acids, hydrostilbene, vitamin A, vitamin E, vitamin C, and red grape extract powder. In addition, these substances may further contain other pharmaceutically, dermatologically and/or cosmetically acceptable media or substrates, such as substances promoting the absorption of protein into the skin, preservatives, hydrating agents, emulsifiers, buffers and so on.

The inventive AIMP1 or its fragment may be administered in an amount of 0.001-20% by weight, and preferably 0.005-10% by weight, based on the total weight of the composition.

The total effective amount of the polypeptide in the inventive composition can be administered to a subject as a single dose, or can be administered using a fractionated treatment protocol, in which the multiple doses are administered over a more prolonged period of time. The amount of the active ingredient in the composition containing the inventive polypeptide may vary depending on the use of the composition, but the active ingredient may be generally administered at an effective dose of 0.1 μg-10 mg several times daily. However, the effective dose of the polypeptide may vary depending on many factors, such as the age, body weight, health condition, sex, disease severity, diet and excretion of a subject in need of treatment, as well as administration time and administration route. In view of these factors, any person skilled in the art may determine an effective dose suitable for the above-described specific use of the inventive polypeptide. The inventive composition has no special limitations on its formulation, administration route and administration mode as long as it shows the effects of the present invention.

### BRIEF DESCRIPTION OF THE DRAWINGS

FIG. 1 shows the results of RT-PCR analysis (upper) and Western blot analysis (lower) for the dose-dependent induction of collagen by the AIMP1 in foreskin fibroblast cells.

GADPH: control group for RT-PCR analysis; and

tubulin: control group for Western blot analysis.

FIG. 2 shows the results of RT-PCR analysis (upper) and Western blot analysis (lower) for the time-dependent induction of collagen by the AIMP1 in foreskin fibroblast cells.

FIG. 3 shows the result of the comparison of the amounts of collagen induced by AIMP1 in the re-epithelialization regions of a wild-type mouse (WT) and a AIMP1-deleted homozygous mouse (Ho) by an immunofluorescent staining.

FIG. 4 shows the results of RT-PCR analysis for the AIMP1-induced KGF expression in foreskin fibroblast cells and U2OS cells.

Cyclin D and GADPH: control groups.

FIG. 5 shows the results of RT-PCR analysis for whether collagen and KGF are induced by fragments (N-terminal end and C-terminal end) of the AIMP1 for varying times.

GADPH: control group.

FIG. 6 shows the results of RT-PCR analysis for the expression levels of collagen and KGF induced in foreskin fibroblast cells by AIMP1, its N-terminal fragment peptides set forth in SEQ ID NO: 2 (amino acids 1-147), SEQ ID NO: 12 (amino acids 6-46), SEQ ID NO: 13 (amino acids 1-46) and SEQ ID NO: 14 (amino acids 1-53), and its C-terminal fragment set forth in SEQ ID NO: 15 (amino acids 193-312).

GADPH: control group

FIG. 7 shows the results of Western blot analysis for the collagen synthesis induced in foreskin fibroblast cells by AIMP1 and its N-terminal fragments set forth in SEQ ID NO: 2 (amino acids 1-147) and SEQ ID NO: 12 (amino acids 6-46).

### BEST MODE FOR CARRYING OUT THE INVENTION

Hereinafter, the present invention will be described in detail by examples. It is to be understood, however, that these examples are for illustrative purpose only and are not construed to limit the scope of the present invention.

### Reference Example 1

### Construction of AIMP1 or its Fragments

An AIMP1 consisting of 312 amino acids (SEQ ID NO: 1), and its N-terminal fragment (1-147; SEQ ID NO: 2) and C-terminal fragment (148-312; SEQ ID NO: 3) were constructed according to the method of Park et al. (Park S. G. et al., J. Biol. Chem., 277:45243-45248, 2002).

### Reference Example 2

### Construction of N-Terminal Deletion Fragments and C-Terminal Deletion Fragment of AIMP1

Each of N-terminal deletion fragments and a C-terminal fragment of AIMP1, i.e., AIMP1-(6-46) (SEQ ID NO: 12), AIMP1-(1-46) (SEQ ID NO: 13), AIMP1-(1-53) (SEQ ID NO: 14), AIMP1-(193-312) (SEQ ID NO: 15), AIMP1-(1-192) (SEQ ID NO: 27) and AIMP1-(6-192) (SEQ ID NO: 28) fragments, was constructed. Each of the fragments was synthesized by PCR using the cDNA of AIMP1 as a template with specific primer sets (see Table 1). The PCR reaction conditions were as follows: pre-denaturation of template DNA by heating at 95° C. for 2 min; and then 25 cycles at 95° C. for 30 sec, 56° C. for 30 sec and 72° C. for 1 min; followed by final extension at 72° C. for 5 min. Each of the PCR products was digested with EcoRI and XhoI and ligated into a pGEX4T3 vector (Amersham Biosciences) digested with the same restriction enzymes. E. coli BL21(DE3) was transformed with the vector and cultured to induce the expression of the peptides. Each of the peptides was expressed as a GST-tag fusion protein and purified on GSH agarose gel. To remove lipopolysaccharide, the protein solutions were dialyzed through pyrogen-free buffer (10 mM potassium phosphate buffer, pH 6.0, 100 mM sodium chloride). After the dialysis, the solution was loaded onto polymyxin resin (Bio-Rd) pre-equilibrated with the same buffer and then incubated for 20 minutes followed by elution, thus preparing each of deletion fragments of AIMP1.

TABLE 1
Primer sets used in consturction of N-terminal
deletion fragments and C-terminal deletion
fragment of AIMP1

| Primer | Sequence | SEQ ID NO |
|---|---|---|
| AIMP1-(6-46) | 5′-cgg aat tcg ctg ttc tga | 16 |
| sense | aga gac tgg agc ag-3′ | |
| AIMP1-(6-46) | 5′-gtc tcg agt tac ttc tct | 17 |
| antisense | tcc ctc aaa gtt gcc tg-3′ | |
| AIMP1-(1-46) | 5′-cgg aat tca tgg caa ata | 18 |
| sense | atg atg ctg ttc tga ag-3′ | |
| AIMP1-(1-46) | 5′-gtc tcg agt tac ttc tct | 19 |
| antisense | tcc ctc aaa gtt gcc-3′ | |
| AIMP1-(1-53) | 5′-cgg aat tca tgg caa ata | 20 |
| sense | atg atg ctg ttc tga ag-3′ | |
| AIMP1-(1-53) | 5′-gtc tcg agt taa gca ttt | 21 |
| antisense | tca act cga agt ttc-3′ | |
| AIMP1- | 5′-cgg aat tcc tgg tga atc | 22 |
| (193-312) | atg ttc ctc ttg aac-3′ | |
| sense | | |
| AIMP1- | 5′-gtc tcg agt tat ttg att | 23 |
| (193-312) | cca ctg ttg ctc atg-3′ | |
| antisense | | |
| AIMP1-(1-192) | 5′-cgg aat tca tgg caa ata | 35 |
| sense | atg atg ctg ttc tga ag-3′ | |
| AIMP1-(1-192) | 5′-gtc tcg agt tag cca ctg | 36 |
| antisense | aca act gtc ctt gg-3′ | |
| AIMP1-(6-192) | 5′-cgg aat tcg ctg ttc tga | 37 |
| sense | aga gac tgg agc ag-3′ | |
| AIMP1-(6-192) | 5′-gtc tcg agt tag cca ctg | 38 |
| antisense | aca act gtc ctt gg-3′ | |

### Example 1

### Stimulation of Collagen Synthesis by AIMP1 Treatment at Varying Concentrations

5<1-1> Culture of Foreskin Fibroblast Cells and AIMP1 Treatment

In order to measure the collagen transcript RNA induced by the AIMP1, foreskin fibroblast cells (5×10⁴ cells/well; obtained from MTT; accession number: MC1232) were cultured in 10% serum-containing DMEM medium on a 6-well plate for 12 hours and then cultured in serum-free medium for about 3 hours. Next, the cultured cells were treated with the AIMP1 (SEQ ID NO: 1) at varying AIMP1 concentrations of 0, 20, 50, 100 and 200 nM for 6 hours (RT-PCR) or 12 hours (Western blot).

<1-2> RT-PCR Analysis

The cells treated with the AIMP1 at varying concentrations in Example <1-1> were collected and dissolved in TRIzol (invitrogen). 10% by weight of chloroform was added to the cell lysate, and mixed well. The mixture was centrifuged at 12,000 g for 15 minutes and the supernatant was collected. Ethanol was added to the collected supernatant to a final concentration of 70%. Then, the supernant was centrifuged at 26,000 g for 5 minutes and the precipitant was collected. The precipitant was dried and dissolved in sterilized distilled water, and the total RNA was extracted. 3 μg of the total RNA was used to prepare cDNA. Then, the cDNA was amplified by PCR using collagen-specific primers (SEQ ID NO: 4 and SEQ ID NO: 5). At this time, for use as a control group, the cDNA was subjected to PCR using GADPH-specific primers (SEQ ID NO: 6 and SEQ ID NO: 7). The PCR reaction consisted of: pre-denaturation of the template DNA at 95° C. for 5 min, followed by 25 cycles of 1 min at 95° C., 1 min at 52° C. and 1 min at 72° C., and then a final extension for 5 min at 72° C.

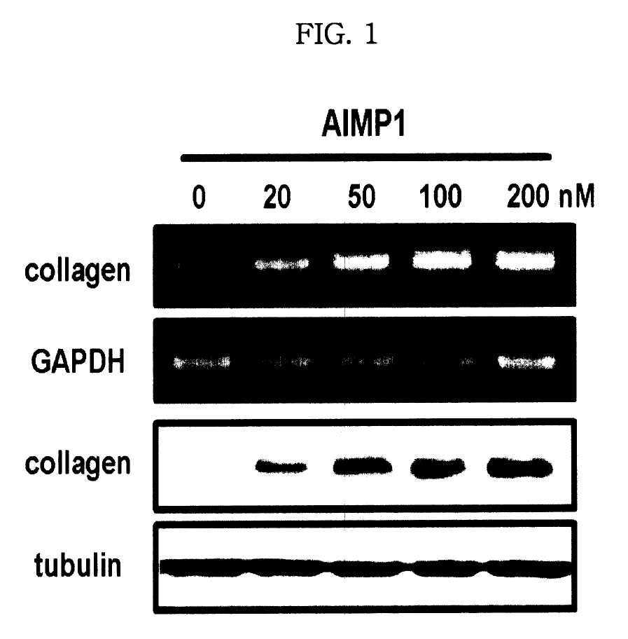

As a result, it could be seen in the upper portion of FIG. 1 that the AIMP1 stimulated the synthesis of collagen in fibroblast cells in dose-dependent manners.

<1-3> Western Blot Analysis

The cells treated with the AIMP1 at varying AIMP1 concentrations in Example <1-1> were collected and lysed in a cell lysis buffer (50 mM HEPES, pH 7.5, 150 mM NaCl, 10% glycerol, 5 mM EGTA, 1 mM sodium orthovanadate, 10 mM NaF, 12 mM b-glycerophosphate, 1 mM DTT, 1 mM PMSF, 5 mg/ml aprotinin, and 1% NP40). Lysed cells were centrifuged at 26,000 g for 15 minutes and the cell extract was isolated. 30 μg of the cell extract was separated by 8% SDS-PAGE and immunoblotted with an anti-collagen I antibody (Abcam) according to any conventional method known in the art. As a control group, an anti-tubulin antibody (Sigma) was used.

As a result, shown in the bottom portion of FIG. 1, it could be seen in the protein levels that the AIMP1 stimulated the synthesis of collagen in fibroblast cells in dose-dependent manners.

### Example 2

### Stimulation of Collagen Synthesis by AIMP1 Treatment at Various Time Intervals

<2-1> Culture of Foreskin Fibroblast Cells and AIMP1 Treatment

Foreskin fibroblast cells (5×10⁴ cells/well) were cultured in 10% serum-containing DMEM medium on a 6-well plate for 12 hours, and then, cultured in serum-free medium for about 3 hours. Next, the cultured cells were treated with 100 nM of the AIMP1 at various time intervals (0, 2, 4, 6, 12 and 24 hours).

<2-2> RT-PCR Analysis

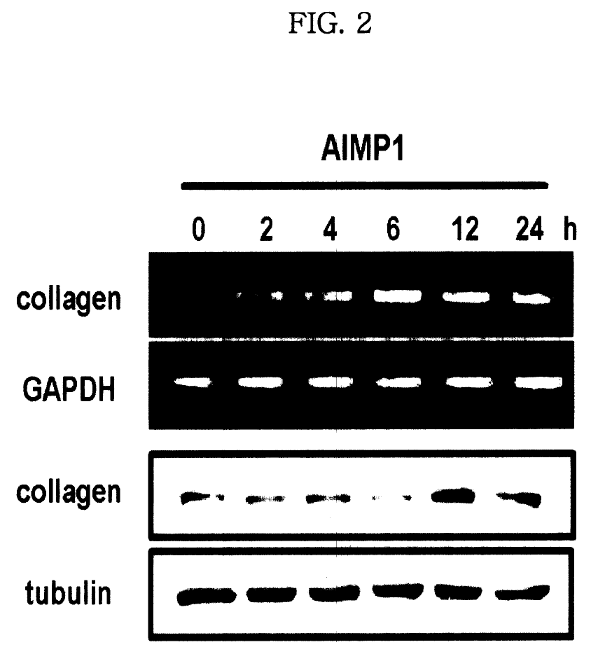

The cells treated with the AIMP1 in Example <2-1> were collected at the specified times and subjected to RT-PCR in the same manner as in Example <1-2>. As a result, it could be seen in the upper portion of FIG. 2 that the AIMP1 stimulated the synthesis of collagen in fibroblast cells in time-dependent manners.

<2-3> Western Blot Analysis

The cells treated with the AIMP1 in Example <2-1> were collected at the specified times and subjected to Western blot analysis in the same manner as in Example <1-3>. As a result, shown in the bottom portion of FIG. 2, it could be seen also in the protein levels that the AIMP1 stimulated the synthesis of collagen.

### Example 3

### Stimulation of Collagen Synthesis by AIMP1 Treatment in In Vivo

By the gene trap method known in the art (Zambrowicz, B. P., et al., Nature 392:608-611, 1998; Kim J Y et al., Proc. Natl. Acad. Sci. USA 99:7912-7916, 2002), a mutation in the AIMP1 gene of a male C57BL6 mouse was induced to prepare a AIMP1 gene deficient homozygous mouse (AIMP1^{-/-} mouse, Ho) (Park S. G. et al., Am. J. Pathol., in press: 2005). Then, on the back of each of the 8-week-old homozygous mouse and wild-type mouse (AIMP1^{+/+} mice, WT), a circular cut-off window with a diameter of 0.5 cm was made with scissors. The wound site was left to stand without dressing. 4 μg of the AIMP1 prepared in Reference Example 1 was dissolved in 5 μl of 20% glycerol-containing PBS and applied on the wound site. The AIMP1 applied twice a day until 3 days after wounding. A control group (AIMP1-untreated group) was treated with 20% glycerol-containing PBS. Then, the tissue of the wound site was isolated using scissors and fixed in 4% paraformaldehyde overnight. The fixed tissue was washed with PBS, incubated in 30% sucrose solution for 4 hours, and then mixed with an OCT (optimal cutting temperature) compound and frozen at −70° C.

6 μm of the frozen section was attached to a silane-coated slide and washed with PBS. Then, the slide was blocked with PBS containing 0.1% tween 20 and 1% non-fat dry milk and allowed to react with an anti-collagen I antibody (Abcam) at 37° C. for 2 hours. The slide was washed three times with PBS containing 0.1% Tween 20, and incubated with a FITC-conjugated secondary antibody at 37° C. for 1 hour. The section was examined under a confocal immunofluorescence microscope (μ-Radiance, BioRad).

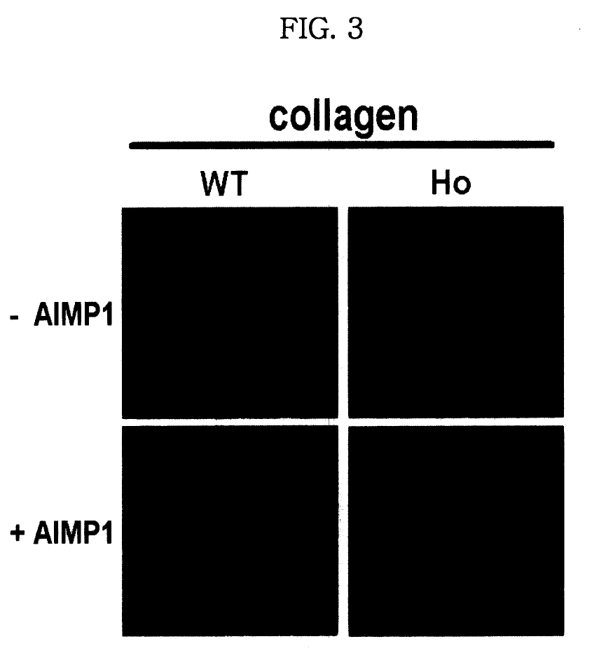

As a result, it could be seen in FIG. 3 that collagen was observed at a larger amount in the wild-type mouse than in the AIMP1 gene deficient homozygous mouse (Ho), and this was further increased by exogenous treatment with the AIMP1. From this, it could be seen also in vivo that the AIMP1 stimulated the synthesis of collagen.

### Example 4

### Stimulation of KGF Expression by AIMP1 Treatment

From foreskin fibroblast cells treated with 100 nM of the AIMP1 for 2 hours, the total RNA was extracted in the same manner as described in Example <1-2>. Then, the extract was amplified by RT-PCR using KGF-specific primers (SEQ ID NO: 8 and SEQ ID NO: 9). The control groups were amplified using cyclin D-specific primers (SEQ ID NO: 10 and SEQ ID NO: 11) and GADPH-specific primers (SEQ ID NO: 6 and SEQ ID NO: 7), respectively. The PCR reaction consisted of: predenaturation of the template DNA at 95° C. for 5 min, followed by 25 cycles of 1 min at 95° C., 1 min at 52° C. and 1 min at 72° C., and then, a final extension for 5 min at 72° C.

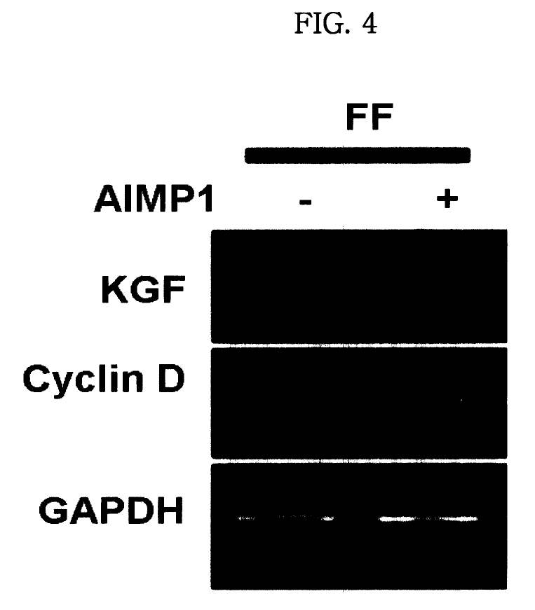

As a result, it could be seen in FIG. 4, the expression of KGF in foreskin fibroblast cells was increased by treatment with the AIMP1. This suggests that the AIMP1 is involved in the regeneration of the skin's epidermal layer by increasing the expression of KGF.

### Example 5

### Analysis of AIMP1 Fragments Activities on Collagen Synthesis and KGF Expression

Foreskin fibroblast cells (5×10⁴ cells/well) were cultured in 10% serum-containing DMEM medium on a 6-well plate about 12 hours, and then, cultured in serum-free medium for about 3 hours. Then, the cultured cells were treated with 100 nM of the N-terminal and C-terminal fragments of the AIMP1 prepared in Reference Example 1, at various time intervals of 0, 1, 2, 4, and 6 hours. Then, RT-PCR was performed in the same manner as in Example <1-2> using each of collagen-specific primers (SEQ ID NO: 4 and SEQ ID NO: 5), KGF-specific primers (SEQ ID NO: 8 and SEQ ID NO: 9) and GADPH-specific primers (SEQ ID NO: 6 and SEQ ID NO: 7).

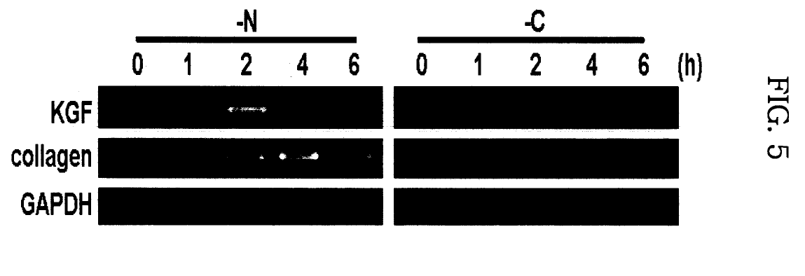

As a result, it was shown in FIG. 5 that the syntheses of collagen and KGF were stimulated by the N-terminal fragment of the AIMP1. However, it was shown that the C-terminal fragment had no significant effect.

### Example 6

### Stimulation of Collagen Synthesis and KGF Expression by N-Terminal Deletion Fragments and C-Terminal Deletion Fragment of AIMP1

Foreskin fibroblast cells (5×10⁴ cells/well) were cultured in 10% serum-containing DMEM medium on a 6-well plate for 12 hours and then cultured in serum-free medium for about 3 hours. Next, the cultured cells were treated with each of the N-terminal fragments (SEQ ID NOS: 12, 13 and 14) and C-terminal fragment (SEQ ID NO: 15) of AIMP1 prepared in Reference Example 2 at an concentration of 100 nM for 2 hours and 6 hours, respectively. The treated cells were subjected to RT-PCR using collagen-specific primers (SEQ ID NOS: 4 and 5), KGF-specific primers (SEQ ID NOS: 8 and 9) and GADPH-specific primers (SEQ ID NOS: 6 and 7) in the same manner as in Example <1-2>.

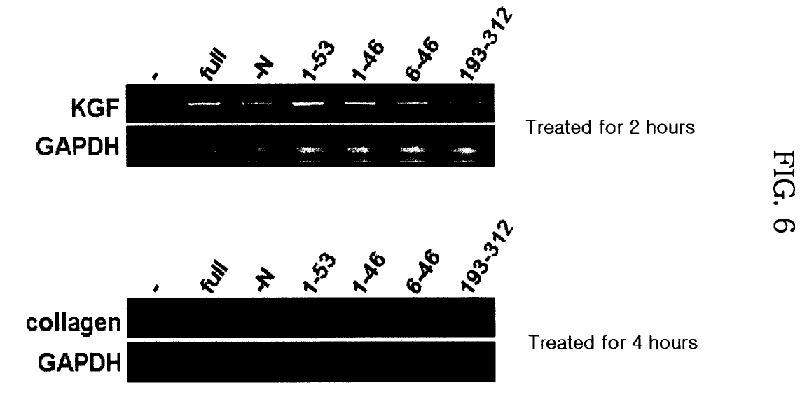

As a result, as shown in FIG. 6, it was observed that, when the cells were treated with the AIMP1 N-terminal deletion fragment (amino acids 6-46; SEQ ID NO: 12) and the peptides comprising thereof (SEQ ID NOs: 13 and 14), collagen synthesis and KGF synthesis were more stimulated than those of the cells treated the AIMP1 C-terminal fragment (amino acids 193-312; SEQ ID NO: 15).

### Example 7

### Stimulation of Collagen Synthesis by AIMP1 and its N-Terminal Fragments

Foreskin fibroblast cells (5×10⁵ cells/well) were cultured in 10% serum-containing DMEM medium on a 6-well plate for 48 hours and then cultured in serum-free medium for about 3 hours. Next, the cultured cells were treated with each of the AIMP1 (SEQ ID NO: 1) and the AIMP1 N-terminal fragments (SEQ ID NOS: 2 and 12) at a concentration of 200 nM for 12 hours. As a control group, cells without treatment were used.

The cells treated with each of the AIMP1 and its N-terminal fragments and the control cells were collected at the indicated time and subjected to Western blot analysis in the same manner as in Example <1-3>.

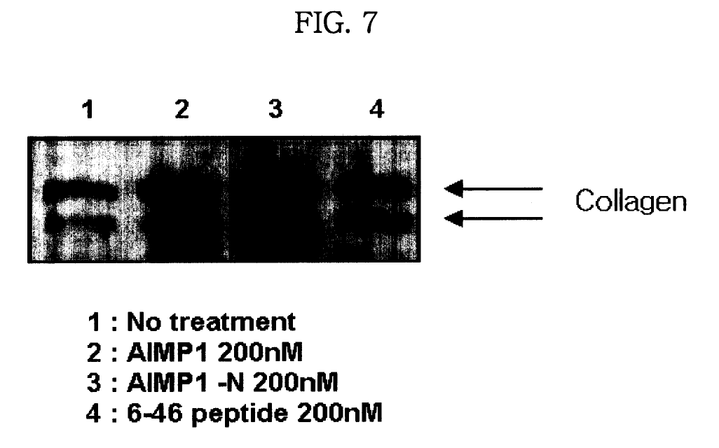

As a result, as shown in FIG. 7, it could be observed in the protein levels that the synthesis of collagen was more stimulated in the foreskin fibroblast cells treated with each of AIMP1 (SEQ ID NO: 1) and its N-terminal fragments (SEQ ID NOs: 2 and 12) than in the foreskin fibroblast cells without AIMP1 treatment.

Formulation 1: Softening Lotion

Softening lotion containing the inventive AIMP1 or its fragment was prepared with components and contents as given in Table 2 below.

TABLE 2

| Component | Content (wt %) |
|---|---|
| Inventive AIMP1 or its fragment | 1.0 |
| Glycerin | 5.0 |
| 1,3-butyleneglycol | 3.0 |
| PEG 1500 | 1.0 |
| Alantoin | 0.1 |
| DL-panthenol | 0.3 |
| EDTA-2Na | 0.02 |
| Benzophenone-9 | 0.04 |
| Sodium hyaluronate | 5.0 |
| Ethanol | 10.0 |
| Octyldodecane-16 | 0.2 |
| Polysorbate 20 | 0.2 |
| Preservative, perfume and pigment | Trace |
| Purified water | Balance |
| Total | 100 |

Formulation 2: Nutrient lotion

Nutrient lotion containing the inventive AIMP1 or its fragment was prepared with components and contents as given in Table 3 below.

TABLE 3

| Component | Content (wt %) |
|---|---|
| Inventive AIMP1 or its fragment | 1.5 |
| Glyceryl stearate SE | 1.5 |
| Stearyl alcohol | 1.5 |
| Lanolin | 1.5 |
| Polysorbate 60 | 1.3 |
| Sorbitan stearate | 0.5 |
| Hydrogenated vegetable oil | 1.0 |
| Mineral oil | 5.0 |
| Squalane | 3.0 |
| Trioctanoin | 2.0 |
| Dimethicone | 0.8 |
| Tocopherol acetate | 0.5 |
| Carboxyvinyl polymer | 0.12 |
| Glycerin | 5.0 |
| 1,3-bytyleneglycol | 3.0 |
| Sodium hyaluronate | 5.0 |
| Triethanolamine | 0.12 |
| Preservative, perfume and pigment | Trace |
| Purified water | Balance |
| Total | 100 |

Formulation 3: Nutrient cream

Nutrient cream containing the inventive AIMP1 or its fragment was prepared with components and contents as given in Table 4 below.

TABLE 4

| Component | Content (wt %) |
|---|---|
| Inventive AIMP1 or its fragment | 2.0 |
| Lipophilic glycerin monostearate | 2.0 |
| Cetearyl alcohol | 2.2 |
| Stearic acid | 1.5 |
| Wax | 1.0 |
| Polysorbate 60 | 1.5 |
| Sorbitan stearate | 0.6 |
| Hydrogenated vegetable oil | 1.0 |
| Squalane | 3.0 |
| Mineral oil | 5.0 |
| Trioctanoin | 5.0 |
| Dimethicone | 1.0 |
| Sodium magnesium silicate | 0.1 |
| Glycerin | 5.0 |
| Betaine | 3.0 |
| Triethanolamine | 1.0 |
| Sodium hyaluronate | 4.0 |
| Preservative, perfume and pigment | Trace |
| Purified water | Balance |
| Total | 100 |

Formulation 4: Essence

Essence containing the inventive AIMP1 or its fragment was prepared with components and contents as given in Table 5 below.

TABLE 5

| Component | Content (wt %) |
|---|---|
| Inventive AIMP1 or its fragment | 2.0 |
| Glycerin | 10.0 |
| Betaine | 5.0 |
| PEG 1500 | 2.0 |
| Alantoin | 0.1 |
| DL-panthenol | 0.3 |
| EDTA-2Na | 0.02 |
| Benzophenone | 0.04 |
| Hydroxyethyl cellulose | 0.1 |
| Sodium hyaluronate | 8.0 |
| Carboxyvinyl polymer | 0.2 |
| Triethanolamine | 0.18 |
| Octyldodecanol | 0.3 |
| Octyldodecane-16 | 0.4 |
| Ethanol | 6.0 |
| Preservative, perfume and pigment | Trace |
| Purified water | Balance |
| Total | 100 |

Formulation 5: Pack

A pack containing the inventive AIMP1 or its fragment was prepared with components and contents as given in Table 6 below.

TABLE 6

| Component | Content (wt %) |
|---|---|
| Inventive AIMP1 or its fragment | 0.5 |
| Glycerin | 5.0 |
| Propylene glycol | 4.0 |
| Polyvinyl alcohol | 15.0 |
| Ethanol | 8.0 |
| Polyoxyethylene oleyl ethyl | 1.0 |
| Methyl paraoxybenzoate | 0.2 |
| Preservative, perfume and pigment | Trace |
| Purified water | Balance |
| Total | 100 |

### INDUSTRIAL APPLICABILITY

As described above, the inventive AIMP1 and its fragment stimulate collagen synthesis and KGF expression in the skin. Accordingly, the inventive AIMP1 and its fragment can be effectively used for the stimulation of collagen synthesis and/or KGF expression in a subject in need thereof, the treatment of skin aging in the subject, the treatment of the flaccid and/or wrinkled skin in the subject, the promoting the skin smoothing and/or firming of the skin in the subject, the treatment of adverse cutaneous effects of menopause in the subject, and the treatment of adverse effects of menopause on collagen.

What is claimed is:

1. A method for stimulating collagen synthesis and/or KGF (keratinocyte growth factor) expression in vitro or in vivo, comprising administering to a subject in need thereof an effective amount of one selected from the group consisting of:
(a) an isolated polypeptide having the amino acid sequence set forth in SEQ ID NO: 1;
(b) an isolated polypeptide having the amino acid sequence homology of at least 70% to the polypeptide (a); and
(c) a fragment of the polypeptide (a) or (b).

2. A method for treating skin aging, comprising administering to a subject in need thereof an effective amount of one selected from the group consisting of:
(a) an isolated polypeptide having an amino acid sequence set forth in SEQ ID NO: 1;
(b) an isolated polypeptide having an amino acid sequence homology of at least 70% with the polypeptide (a); and
(c) a fragment of the polypeptide (a) or (b).

3. A method for treating the flaccid and/or wrinkled skin, comprising administering to a subject in need thereof an effective amount of one selected from the group consisting of:
(a) an isolated polypeptide having the amino acid sequence set forth in SEQ ID NO: 1;
(b) an isolated polypeptide having the amino acid sequence homology of at least 70% with the polypeptide (a); and
(c) a fragment of the polypeptide (a) or (b).

4. A method for promoting the smoothing and/or firming of the skin, comprising administration to a subject in need thereof of an effective amount of one selected from the group consisting of:
(a) an isolated polypeptide having the amino acid sequence set forth in SEQ ID NO: 1;
(b) an isolated polypeptide having the amino acid sequence homology of at least 70% with the polypeptide (a); and
(c) a fragment of the polypeptide (a) or (b).

5. A method for treating adverse cutaneous effects of menopause, comprising administration to a subject in need thereof of an effective amount of one selected from the group consisting of:
(a) an isolated polypeptide having the amino acid sequence set forth in SEQ ID NO: 1;
(b) an isolated polypeptide having the amino acid sequence homology of at least 70% with the polypeptide (a); and
(c) a fragment of the polypeptide (a) or (b).

6. A method for treating adverse effects of menopause on the collagen, comprising administration to a subject in need thereof of an effective amount of one selected from the group consisting of:
(a) an isolated polypeptide having the amino acid sequence set forth in SEQ ID NO: 1;
(b) an isolated polypeptide having the amino acid sequence homology of at least 70% with the polypeptide (a); and
(c) a fragment of the polypeptide (a) or (b).

7. The method of any one of claims 1 to 6, wherein the polypeptide of (b) is selected from the group consisting of SEQ ID NO: 24, SEQ ID NO: 25 and SEQ ID NO: 26.

8. The method of any one of claims 1 to 6, wherein the fragment of (c) is a peptide comprising the amino acid sequence set forth in SEQ ID NO: 12 and consisting of at least 192 amino acids.

9. The method of claim 8, wherein the fragment of (c) is a peptide consisting of the amino acid sequence selected from SEQ ID NO: 2, SEQ ID NO: 12, SEQ ID No: 13, SEQ ID NO: 14 and SEQ ID NOs: 27 to 34.

10. A composition for stimulating collagen synthesis and/or KGF expression, the composition comprising as an active ingredient one selected from the group consisting of:
(a) an isolated polypeptide having the amino acid sequence set forth in SEQ ID NO: 1;
(b) an isolated polypeptide having the amino acid sequence homology of at least 70% with the polypeptide (a); and
(c) a fragment of the polypeptide (a) or (b).

11. A cosmetic composition for preventing skin aging, the composition comprising as an active ingredient one selected from the group consisting of:
(a) an isolated polypeptide having the amino acid sequence set forth in SEQ ID NO: 1;
(b) an isolated polypeptide having the amino acid sequence homology of at least 70% with the polypeptide (a); and
(c) a fragment of the polypeptide (a) or (b).

12. The composition of claim 10 or claim 11, wherein the polypeptide of (b) is selected from the group consisting of SEQ ID NO: 24, SEQ ID NO: 25 and SEQ ID NO: 26.

13. The composition of claim 10 or claim 11, wherein the fragment of (c) is a peptide comprising the amino acid sequence set forth in SEQ ID NO: 12 and consisting of at least 192 amino acids.

14. The composition of claim 13, wherein the fragment of (c) is a peptide consisting of the amino acid sequence selected from SEQ ID NO: 2, SEQ ID NO: 12, SEQ ID No: 13, SEQ ID NO: 14 and SEQ ID NOs: 27 to 34.

15. Use of any one selected from the group consisting of the following, for preparing a composition for stimulating collagen synthesis and/or KGF expression, the composition comprising the one as an active ingredient:
(a) an isolated polypeptide having the amino acid sequence set forth in SEQ ID NO:1;
(b) an isolated polypeptide having the amino acid sequence homology of at least 70% with the polypeptide (a); and
(c) a fragment of the polypeptide (a) or (b).

16. Use of any one selected from the group consisting of the following proteins, for preparing a cosmetic composition for preventing skin aging, the composition comprising the one as an active ingredient:
(a) an isolated polypeptide having the amino acid sequence set forth in SEQ ID NO: 1;
(b) an isolated polypeptide having the amino acid sequence homology of at least 70% with the polypeptide (a); and
(c) a fragment of the polypeptide (a) or (b).

17. The use of claim 15 or claim 16, wherein the polypeptide of (b) is selected from the group consisting of SEQ ID NO: 24, SEQ ID NO: 25 and SEQ ID NO: 26.

18. The use of claim 15 or claim 16, wherein the fragment of (c) is a peptide comprising the amino acid sequence set forth in SEQ ID NO: 12 and consisting of at least 192 amino acids.

19. The use of claim 18, wherein the fragment of (c) is a peptide consisting of the amino acid sequence selected from SEQ ID NO: 2, SEQ ID NO: 12, SEQ ID No: 13, SEQ ID NO: 14 and SEQ ID NOs: 27 to 34.


SEQUENCE LISTING (38 sequences)

SEQ ID NO: 1 - protein, 312 aa, Homo sapiens
MANNDAVLKRLEQKGAEADQIIEYLKQQVSLLKEKAILQATLREEKKLRVENAKLKKEIEELKQELIQAEIQNGVKQIAFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSGLVNHVPLEQMQNRMVILLCNLKPAKMRGVLSQAMVMCASSPEKIEILAPPNGSVPGDRITFDAFPGEPDKELNPKKKIWEQIQPDLHTNDECVATYKGVPFEVKGKGVCRAQTMSNSGIK

SEQ ID NO: 2 - protein, 147 aa, Homo sapiens
  PEPTIDE (1)..(147): AIMP1-(1-147)
MANNDAVLKRLEQKGAEADQIIEYLKQQVSLLKEKAILQATLREEKKLRVENAKLKKEIEELKQELIQAEIQNGVKQIAFPSGTPLHANSMVSENVIQSTAVTTVSSGTKEQIKGGTGDEKKAKEKIEKKGEKKEKKQQSIAGSADS

SEQ ID NO: 3 - protein, 165 aa, Homo sapiens
  PEPTIDE (1)..(165): AIMP1-(148-312)
KPIDVSRLDLRIGCIITARKHPDADSLYVEEVDVGEIAPRTVVSGLVNHVPLEQMQNRMVILLCNLKPAKMRGVLSQAMVMCASSPEKIEILAPPNGSVPGDRITFDAFPGEPDKELNPKKKIWEQIQPDLHTNDECVATYKGVPFEVKGKGVCRAQTMSNSGIK

SEQ ID NO: 4 - DNA, 21 bp, Artificial Sequence
  forward primer for collagen
gtcttcctggcccctctggtg

SEQ ID NO: 5 - DNA, 21 bp, Artificial Sequence
  reverse primer for collagen
tcgccctgttcgcctgtctca

SEQ ID NO: 6 - DNA, 21 bp, Artificial Sequence
  forward primer for GADPH
tttggtcgtattgggcgcctg

SEQ ID NO: 7 - DNA, 21 bp, Artificial Sequence
  reverse primer for GADPH
ccatgacgaacatgggggcat

SEQ ID NO: 8 - DNA, 21 bp, Artificial Sequence
  forward primer for KGF
gagcactacactataatgcac

SEQ ID NO: 9 - DNA, 21 bp, Artificial Sequence
  reverse primer for KGF
agttattgccataggaagaag

SEQ ID NO: 10 - DNA, 21 bp, Artificial Sequence
  forward primer for cyclin-D
atggagctgctgtgccacgag

SEQ ID NO: 11 - DNA, 21 bp, Artificial Sequence
  reverse primer for cyclin-D
caggtcgatatcccgcacgtc

SEQ ID NO: 12 - protein, 41 aa, Homo sapiens
  PEPTIDE (1)..(41): AIMP1-(6-46)
AVLKRLEQKGAEADQIIEYLKQQVSLLKEKAILQATLREEK

SEQ ID NO: 13 - protein, 46 aa, Homo sapiens
  PEPTIDE (1)..(46): AIMP1-(1-46)
MANNDAVLKRLEQKGAEADQIIEYLKQQVSLLKEKAILQATLREEK

SEQ ID NO: 14 - protein, 53 aa, Homo sapiens
  PEPTIDE (1)..(53): AIMP1-(1-53)
MANNDAVLKRLEQKGAEADQIIEYLKQQVSLLKEKAILQATLREEKKLRVENA

SEQ ID NO: 15 - protein, 120 aa, Homo sapiens
  PEPTIDE (1)..(120): AIMP1-(193-312)
LVNHVPLEQMQNRMVILLCNLKPAKMRGVLSQAMVMCASSPEKIEILAPPNGSVPGDRITFDAFPGEPDKELNPKKKIWEQIQPDLHTNDECVATYKGVPFEVKGKGVCRAQTMSNSGIK

SEQ ID NO: 16 - DNA, 32 bp, Artificial Sequence
  forward primer for AIMP1(6-46)
cggaattcgctgttctgaagagactggagcag

SEQ ID NO: 17 - DNA, 35 bp, Artificial Sequence
  reverse primer for AIMP1(6-46)
gtctcgagttacttctcttccctcaaagttgcctg

SEQ ID NO: 18 - DNA, 35 bp, Artificial Sequence
  forward primer for AIMP1(1-46)
cggaattcatggcaaataatgatgctgttctgaag

SEQ ID NO: 19 - DNA, 33 bp, Artificial Sequence
  reverse primer for AIMP1(1-46)
gtctcgagttacttctcttccctcaaagttgcc

SEQ ID NO: 20 - DNA, 35 bp, Artificial Sequence; identical to SEQ ID NO: 18
  forward primer for AIMP1(1-53)

SEQ ID NO: 21 - DNA, 33 bp, Artificial Sequence
  reverse primer for AIMP1(1-53)
gtctcgagttaagcattttcaactcgaagtttc

SEQ ID NO: 22 - DNA, 33 bp, Artificial Sequence
  forward primer for AIMP1-(193-312)
cggaattcctggtgaatcatgttcctcttgaac

SEQ ID NO: 23 - DNA, 33 bp, Artificial Sequence
  reverse primer for AIMP1-(193-312)
gtctcgagttatttgattccactgttgctcatg

SEQ ID NO: 24 - protein, 312 aa, Homo sapiens
  PEPTIDE (1)..(312): AIMP1-SNP(79-Pro)
MANNDAVLKRLEQKGAEADQIIEYLKQQVSLLKEKAILQATLREEKKLRVENAKLKKEIEELKQELIQAEIQNGVKQIPFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSGLVNHVPLEQMQNRMVILLCNLKPAKMRGVLSQAMVMCASSPEKIEILAPPNGSVPGDRITFDAFPGEPDKELNPKKKIWEQIQPDLHTNDECVATYKGVPFEVKGKGVCRAQTMSNSGIK

SEQ ID NO: 25 - protein, 312 aa, Homo sapiens
  PEPTIDE (1)..(312): AIMP1-SNP(104-Ala)
MANNDAVLKRLEQKGAEADQIIEYLKQQVSLLKEKAILQATLREEKKLRVENAKLKKEIEELKQELIQAEIQNGVKQIAFPSGTPLHANSMVSENVIQSTAVTAVSSGTKEQIKGGTGDEKKAKEKIEKKGEKKEKKQQSIAGSADSKPIDVSRLDLRIGCIITARKHPDADSLYVEEVDVGEIAPRTVVSGLVNHVPLEQMQNRMVILLCNLKPAKMRGVLSQAMVMCASSPEKIEILAPPNGSVPGDRITFDAFPGEPDKELNPKKKIWEQIQPDLHTNDECVATYKGVPFEVKGKGVCRAQTMSNSGIK

SEQ ID NO: 26 - protein, 312 aa, Homo sapiens
  PEPTIDE (1)..(312): AIMP1-SNP(117-Ala)
MANNDAVLKRLEQKGAEADQIIEYLKQQVSLLKEKAILQATLREEKKLRVENAKLKKEIEELKQELIQAEIQNGVKQIAFPSGTPLHANSMVSENVIQSTAVTTVSSGTKEQIKGGAGDEKKAKEKIEKKGEKKEKKQQSIAGSADSKPIDVSRLDLRIGCIITARKHPDADSLYVEEVDVGEIAPRTVVSGLVNHVPLEQMQNRMVILLCNLKPAKMRGVLSQAMVMCASSPEKIEILAPPNGSVPGDRITFDAFPGEPDKELNPKKKIWEQIQPDLHTNDECVATYKGVPFEVKGKGVCRAQTMSNSGIK

SEQ ID NO: 27 - protein, 192 aa, Homo sapiens
  PEPTIDE (1)..(192): AIMP1-(1-192)
MANNDAVLKRLEQKGAEADQIIEYLKQQVSLLKEKAILQATLREEKKLRVENAKLKKEIEELKQELIQAEIQNGVKQIAFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSG

SEQ ID NO: 28 - protein, 187 aa, Homo sapiens
  PEPTIDE (1)..(187): AIMP1-(6-192)
AVLKRLEQKGAEADQIIEYLKQQVSLLKEKAILQATLREEKKLRVENAKLKKEIEELKQELIQAEIQNGVKQIAFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSG

SEQ ID NO: 29 - protein, 192 aa, Homo sapiens
MANNDAVLKRLEQKGAEADQIIEYLKQQVSLLKEKAILQATLREEKKLRVENAKLKKEIEELKQELIQAEIQNGVKQIPFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSG

SEQ ID NO: 30 - protein, 192 aa, Homo sapiens
MANNDAVLKRLEQKGAEADQIIEYLKQQVSLLKEKAILQATLREEKKLRVENAKLKKEIEELKQELIQAEIQNGVKQIAFPSGTPLHANSMVSENVIQSTAVTAVSSGTKEQIKGGTGDEKKAKEKIEKKGEKKEKKQQSIAGSADSKPIDVSRLDLRIGCIITARKHPDADSLYVEEVDVGEIAPRTVVSG

SEQ ID NO: 31 - protein, 192 aa, Homo sapiens
MANNDAVLKRLEQKGAEADQIIEYLKQQVSLLKEKAILQATLREEKKLRVENAKLKKEIEELKQELIQAEIQNGVKQIAFPSGTPLHANSMVSENVIQSTAVTTVSSGTKEQIKGGAGDEKKAKEKIEKKGEKKEKKQQSIAGSADSKPIDVSRLDLRIGCIITARKHPDADSLYVEEVDVGEIAPRTVVSG

SEQ ID NO: 32 - protein, 187 aa, Homo sapiens
AVLKRLEQKGAEADQIIEYLKQQVSLLKEKAILQATLREEKKLRVENAKLKKEIEELKQELIQAEIQNGVKQIPFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSG

SEQ ID NO: 33 - protein, 187 aa, Homo sapiens
AVLKRLEQKGAEADQIIEYLKQQVSLLKEKAILQATLREEKKLRVENAKLKKEIEELKQELIQAEIQNGVKQIAFPSGTPLHANSMVSENVIQSTAVTAVSSGTKEQIKGGTGDEKKAKEKIEKKGEKKEKKQQSIAGSADSKPIDVSRLDLRIGCIITARKHPDADSLYVEEVDVGEIAPRTVVSG

SEQ ID NO: 34 - protein, 187 aa, Homo sapiens
AVLKRLEQKGAEADQIIEYLKQQVSLLKEKAILQATLREEKKLRVENAKLKKEIEELKQELIQAEIQNGVKQIAFPSGTPLHANSMVSENVIQSTAVTTVSSGTKEQIKGGAGDEKKAKEKIEKKGEKKEKKQQSIAGSADSKPIDVSRLDLRIGCIITARKHPDADSLYVEEVDVGEIAPRTVVSG

SEQ ID NO: 35 - DNA, 35 bp, Artificial Sequence; identical to SEQ ID NO: 18
  forward primer for AIMP1-(1-192)

SEQ ID NO: 36 - DNA, 32 bp, Artificial Sequence
  reverse primer for AIMP1-(1-192)
gtctcgagttagccactgacaactgtccttgg

SEQ ID NO: 37 - DNA, 32 bp, Artificial Sequence; identical to SEQ ID NO: 16
  forward primer for AIMP1-(6-192)

SEQ ID NO: 38 - DNA, 32 bp, Artificial Sequence; identical to SEQ ID NO: 36
  reverse primer for AIMP1-(6-192)